(19) United States
(12) Patent Application Publication
(10) Pub. No.: US 2025/0247940 A1
(43) Pub. Date: Jul. 31, 2025

(54) X-Ray Imaging Apparatus and Method for Adjusting Current to Be Supplied to Filament

(71) Applicant: SHIMADZU CORPORATION, Kyoto-shi (JP)

(72) Inventors: Ryo TATENO, Kyoto-shi (JP); Tsuyoshi NARITA, Kyoto-shi (JP)

(21) Appl. No.: 19/031,445

(22) Filed: Jan. 18, 2025

(30) Foreign Application Priority Data
Jan. 26, 2024 (JP) 2024-010438

(51) Int. Cl. H05G 1/46; G01N 23/04; H01J 35/06; H05G 1/26; H05G 1/36

(52) U.S. Cl. CPC H05G 1/46; G01N 23/04; H01J 35/064; H05G 1/265; H05G 1/36

(57) ABSTRACT

An X-ray imaging apparatus includes an X-ray tube including an electron emitter including a filament, a storage to store current information acquired in advance, in which a total supply time of a current to be supplied to the filament is associated with an appropriate current value to be supplied to the filament, and a controller configured or programmed to perform a control to adjust the current to be supplied to the filament based on the current information after a first total supply time as a predetermined total supply time.

### CROSS-REFERENCE TO RELATED APPLICATIONS

This application claims the benefit of priority to Japanese Patent Application No. 2024-010438 filed on Jan. 26, 2024. The entire contents of this application are hereby incorporated herein by reference.

### BACKGROUND OF THE INVENTION

### Field of the Invention

The present invention relates to an X-ray imaging apparatus and a method for adjusting a current to be supplied to a filament.

### Description of the Background Art

Conventionally, an X-ray imaging apparatus is known. Such an apparatus is disclosed in Japanese Patent Laid-Open No. 2008-251300, for example.

Japanese Patent Laid-Open No. 2008-251300 discloses an X-ray inspection device (X-ray imaging apparatus) including an X-ray tube, an X-ray detector, and a controller. The X-ray tube includes a target and an electron emitter including a filament. In a vacuum vessel, the X-ray tube converges an electron beam emitted from the filament by a magnetic lens to strike the target, and radiates X-rays generated in the target. A current is supplied to the filament to heat the filament such that the filament emits thermoelectrons. In X-ray inspection, when the current supplied to the filament is too small, a sufficient tube current cannot be obtained, and a desired X-ray brightness cannot be obtained. In contrast, when the current supplied to the filament is too large, the filament is cut due to the progression of filament burnout and becomes unusable such that the life of the filament is shortened. Japanese Patent Laid-Open No. 2008-251300 discloses that, before the X-ray inspection is performed, a current value (saturated filament current) is searched for when the brightness value based on an X-ray detection signal stops changing and reaches a saturation region while the current to be supplied to the filament is sequentially increased, and the X-ray inspection is performed by supplying, to the filament, a current that is equal to or less than the searched saturated filament current value.

However, even in a process to search for the saturated filament current before performing the X-ray inspection, a current is supplied to the filament. Therefore, even in the above process, the filament burnout progresses such that the life of the filament is shortened. The inventors of the present invention have found that, with regard to the filament cutting caused by the progression of filament burnout, the filament is cut more often in the above process than in X-ray imaging that is performed by supplying, to a filament, a current equal to or less than a searched saturated filament current value.

The inventors of the present invention have then found a problem of reducing or preventing shortening of the usable life of the filament due to the process to search for an appropriate current value to be supplied to the filament.

### SUMMARY OF THE INVENTION

The present invention is intended to solve the above problem. The present invention aims to provide an X-ray imaging apparatus capable of reducing or preventing shortening of the usable life of a filament due to a process to search for an appropriate current value to be supplied to the filament, and a method for adjusting a current to be supplied to a filament.

In order to attain the aforementioned object, an X-ray imaging apparatus according to a first aspect of the present invention includes an X-ray tube including a target and an electron emitter including a filament to emit electrons to the target, an X-ray detector to detect X-rays emitted from the X-ray tube, a storage to store current information acquired in advance, in which a total supply time of a current to be supplied to the filament to emit the electrons from the filament is associated with an appropriate current value to be supplied to the filament to emit the electrons from the filament, and a controller configured or programmed to perform a control to adjust the current to be supplied to the filament based on the current information after a first total supply time as a predetermined total supply time.

In order to attain the aforementioned object, a method for adjusting a current to be supplied to a filament according to a second aspect of the present invention is a method for adjusting a current to be supplied to a filament in an X-ray imaging apparatus including an X-ray tube including a target and an electron emitter including a filament to emit electrons to the target, and includes acquiring current information in advance, in which a total supply time of the current to be supplied to the filament to emit the electrons from the filament is associated with an appropriate current value to be supplied to the filament to emit the electrons from the filament, and adjusting the current to be supplied to the filament based on the current information after a first total supply time as a predetermined total supply time.

In the X-ray imaging apparatus according to the first aspect and the method for adjusting the current to be supplied to the filament according to the second aspect, the current information is acquired in advance, in which the total supply time of the current to be supplied to the filament to emit the electrons from the filament is associated with the appropriate current value to be supplied to the filament to emit the electrons from the filament, and after the first total supply time as the predetermined total supply time, the current to be supplied to the filament is adjusted based on the current information. Accordingly, after the first total supply time, the current to be supplied to the filament is adjusted based on the current information acquired in advance, without performing the process to acquire the appropriate current value to be supplied to the filament by supplying a current to the filament. Therefore, after the first total supply time, the process to supply a current to the filament to search for an appropriate current value is not performed, and thus a current supply to the filament by the above process can be reduced or prevented. Consequently, it is possible to reduce or prevent shortening of the usable life of the filament due to the above process. Furthermore, after the first total supply time, the current to be supplied to the filament can be appropriately adjusted based on the current information acquired in advance without performing the above process.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view showing the overall configuration of an X-ray imaging apparatus according to an embodiment.

FIG. 2 is a schematic view showing the configuration of an X-ray tube according to the embodiment.

FIG. 3 is a graph for illustrating an appropriate current value acquired in a process to search for the appropriate current value.

FIG. 4 is a graph for illustrating an example of current information.

FIG. 5 is a flowchart for illustrating a control to adjust a current to be supplied to a filament.

### DESCRIPTION OF THE PREFERRED EMBODIMENTS

An embodiment of the present invention is hereinafter described with reference to the drawings.

### Configuration of X-Ray Imaging Apparatus

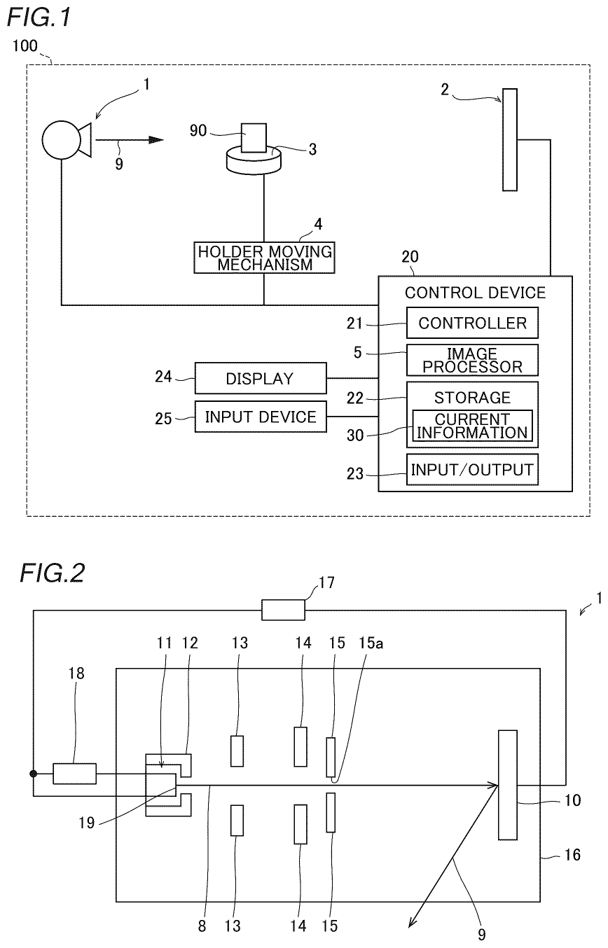

The configuration of an X-ray imaging apparatus 100 according to this embodiment is now described with reference to FIGS. 1 to 4.

As shown in FIG. 1, the X-ray imaging apparatus 100 is an apparatus that captures an X-ray image of a subject 90. The X-ray imaging apparatus 100 according to this embodiment is used for non-destructive inspection of the subject 90, for example. In such a case, the subject 90 is a sample to be inspected.

The X-ray imaging apparatus 100 includes an X-ray tube 1, an X-ray detector 2, a subject holder 3, a holder moving mechanism 4, and a control device 20.

The X-ray tube 1 irradiates the subject 90 placed on the subject holder 3 with X-rays 9. The X-ray tube 1 generates the X-rays 9 when a high voltage is applied to the X-ray tube 1. The X-ray tube 1 faces the X-ray detector 2 via the subject holder 3. In this embodiment, the X-ray tube 1, the subject holder 3, and the detector are arranged side by side in a horizontal direction.

As shown in FIG. 2, the X-ray tube 1 includes an electron emitter 11 serving as a cathode, a grid electrode 12, an anode 13, an electron lens 14, an aperture 15, a target 10, a vacuum vessel 16, a first power supply circuit 17, and a second power supply circuit 18. The electron emitter 11, the grid electrode 12, the anode 13, the electron lens 14, and the target 10 are housed in the vacuum vessel 16.

The electron emitter 11 emits an electron beam 8 to the target 10. The electron emitter 11 generates electrons when a current is applied from the second power supply circuit 18 to the electron emitter 11. The electron emitter 11 also emits the generated electrons toward the target 10 to which a high voltage is applied. The electron emitter 11 includes a filament 19 in which heavy metal is formed into a coil or foil shape, for example. The filament 19 is made of tungsten, for example.

A predetermined voltage is applied between the electron emitter 11 and the target 10 by the first power supply circuit 17. Specifically, the electron emitter 11 and the target 10 are connected to the first power supply circuit 17 via wiring. The electron emitter 11 is also connected to the second power supply circuit 18 via wiring. The filament 19 of the electron emitter 11 is heated by being energized by the second power supply circuit 18. Thus, the electron beam 8 (thermal electrons) is generated from the electron emitter 11 toward the target 10. Furthermore, a current to be supplied to the filament 19 is adjusted by a controller 21 via the second power supply circuit 18.

The grid electrode 12 controls the amount of current of the emitted electron beam 8. The grid electrode 12 is provided in the vicinity of the electron emitter 11.

The anode 13 accelerates the electrons emitted from the electron emitter 11 when a voltage is applied the anode 13. The anode 13 is provided between the electron emitter 11 and the target 10.

The electron lens 14 converges the electron beam 8 emitted from the electron emitter 11. The electron lens 14 causes the electron beam 8 emitted from the electron emitter 11 to be incident on a surface of the target 10 substantially perpendicularly. The electron lens 14 is provided between the electron emitter 11 and the target 10. In this embodiment, the electron lens 14 is an electromagnetic lens. The electromagnetic lens is an electromagnet using a coil, and has magnetic poles (pole pieces) (not shown) protruding toward the center of the coil hole. The electrons emitted from the electron emitter 11 pass through a range (hole) surrounded by the pole pieces. The electron lens 14 configured to converge the electron beam 8 may be an electrostatic lens instead of an electromagnetic lens, or may be another known lens.

The aperture 15 can adjust the passing range of the electrons. The aperture 15 includes a diaphragm aperture 15a. The aperture 15 is provided between the electron emitter 11 and the target 10. The electrons emitted from the electron emitter 11 and passing through the diaphragm aperture 15a of the aperture 15 collide with the target 10.

The target 10 generates X-rays 9 when the electron beam 8 (thermal electrons) emitted from the electron emitter 11 collides with the target 10. The target 10 is made of a metal material such as tungsten, molybdenum, copper, cobalt, chromium, iron, or silver. The target 10 is a reflective target. The reflective target is a type of target in which X-rays 9 are radiated so as to be reflected by the surface in a direction different from a direction in which the electron beam 8 comes. The target 10 may be a transmissive target. The transmissive target includes a pair of (front and back) surfaces perpendicular to the electron beam 8, and is a type of target in which when electrons collide with one surface, X-rays 9 are radiated from the other surface such that the X-rays 9 pass through the target 10.

The electron emitter 11 and the target 10 are arranged inside the vacuum vessel 16. The inside of the vacuum vessel 16 is sealed in a substantially vacuum state. The vacuum vessel 16 is made of a non-magnetic metal material such as stainless steel (SUS). The vacuum vessel 16 includes a window (not shown) through which X-rays 9 are radiated to the outside.

As shown in FIG. 1, the X-ray detector 2 detects X-rays 9 radiated from the X-ray tube 1. The X-rays 9 radiated from the X-ray tube 1 pass through the subject 90 and enter a detection surface of the X-ray detector 2. The X-ray detector 2 converts the detected X-rays 9 into electric signals. Thus, an X-ray image reflecting the transmission of the X-rays 9 through the subject 90 is obtained. The X-ray detector 2 is a flat panel detector (FPD), for example. The X-ray detector 2 includes a plurality of conversion elements (not shown) and pixel electrodes (not shown) arranged on the plurality of conversion elements. The plurality of conversion elements and the pixel electrodes are arranged in a matrix on the detection surface at predetermined cycles (pixel pitches). Detection signals (image signals) of the X-ray detector 2 are transmitted to an image processor 5.

The subject holder 3 is arranged between the X-ray tube 1 and the X-ray detector 2, and supports the subject 90. The subject holder 3 includes a stage on which the subject 90 is placed. The subject 90 may be placed on the subject holder 3 via a retainer (not shown) configured to retain the subject 90, for example.

The holder moving mechanism 4 moves the subject holder 3 in two mutually perpendicular directions in a horizontal plane or with respect to a vertical direction. The holder moving mechanism 4 includes a motor (not shown) configured to move the subject holder 3, which is a stage.

The control device 20 includes a personal computer (PC), for example, and includes the controller 21, the image processor 5, a storage 22, and an input/output 23. The control device 20 is connected to a display 24 and an input device 25.

The controller 21 includes a processor such as a central processing unit (CPU), and executes application programs stored in the storage 22 to set imaging conditions in the X-ray imaging apparatus 100, control the start and stop of imaging, control the operation of the X-ray tube 1, and control a process to search for an appropriate current value. The above process is described below in detail.

The controller 21 adjusts a current to be supplied to the filament 19 based on current information 30 after a first total supply time as a predetermined total supply time. Specifically, the controller 21 adjusts the current to be supplied to the filament 19 based on an appropriate current value acquired by the process to search for an appropriate current value for up to the first total supply time, and adjusts the current to be supplied to the filament 19 based on the current information 30 during a second total supply time after the first total supply time. The current information 30 and a control to adjust the current to be supplied to the filament 19 based on the current information 30 are described below in detail.

The image processor 5 includes a processor such as a graphics processing unit (GPU) or a field-programmable gate array (FPGA) configured for image processing. The image processor 5 acquires X-ray image data from the X-ray detector 2. The image processor 5 performs a predetermined image process on the acquired X-ray image data.

The storage 22 includes a volatile storage and a non-volatile storage. The storage 22 stores various programs. The storage 22 also stores previously acquired current information 30, an offset value table, and a correction value table. In this description, “previously acquired” in “previously acquired current information 30” does not refer to “acquired before the start of the first X-ray inspection after manufacture of the X-ray imaging apparatus 100 or after shipment of the X-ray imaging apparatus 100”, but refers to, for example, “acquired by a manufacturer of the X-ray imaging apparatus 100 through an experiment in common for each model or product number of the X-ray imaging apparatus 100”. The offset value table and the correction value table are described below in detail.

The input/output 23 includes various interfaces configured to input and output signals to and from the control device 20. The input/output 23 is connected to the display 24 and the input device 25. The display 24 is a liquid crystal display, for example. The input device 25 includes a keyboard, a mouse, etc. The image processor 5 acquires detection signals (image signals) from the X-ray detector 2 via the input/output 23.

### Process to Search for Appropriate Current Value

The process to search for an appropriate current value is now described.

In X-ray imaging, it is important to set the current to be supplied to the filament 19 to an appropriate value in order to obtain an image with a desired X-ray brightness. When the current to be supplied to the filament 19 is too small, a sufficient tube current is not obtained, and the desired X-ray brightness is not obtained. In contrast, when the current to be supplied to the filament 19 is too large, the filament 19 is cut due to the progression of burnout of the filament 19 to be unusable such that the life of the filament 19 is shortened. Therefore, the controller 21 performs a process to acquire an appropriate current value to be supplied to the filament 19 by supplying a current to the filament 19. The above process is performed at each predetermined third total supply time interval that is set in advance. The total supply time refers to the total supply time of the current to be supplied to the filament 19 to emit electrons from the filament 19. The above process may be performed based on an input operation by a user.

The controller 21 acquires an appropriate current value by the process to search for an appropriate current value for up to the first total supply time from the start of use of the filament 19. An oxide film is formed on the filament 19 before the start of use. Due to the formed oxide film, the appropriate current value is not stable in the filament 19 immediately after the start of use. The controller 21 performs the above process for up to the first total supply time, and acquires an appropriate current value to be supplied to the filament 19 by supplying a current to the filament 19. The first total supply time refers to a predicted total supply time required for a fluctuation in the appropriate current value acquired by the above process to stabilize due to the oxide film of the filament 19 peeling off with use. The first total supply time is 96 hours, for example. The first total supply time is not limited to 96 hours, but may be shorter than 96 hours or longer than 96 hours.

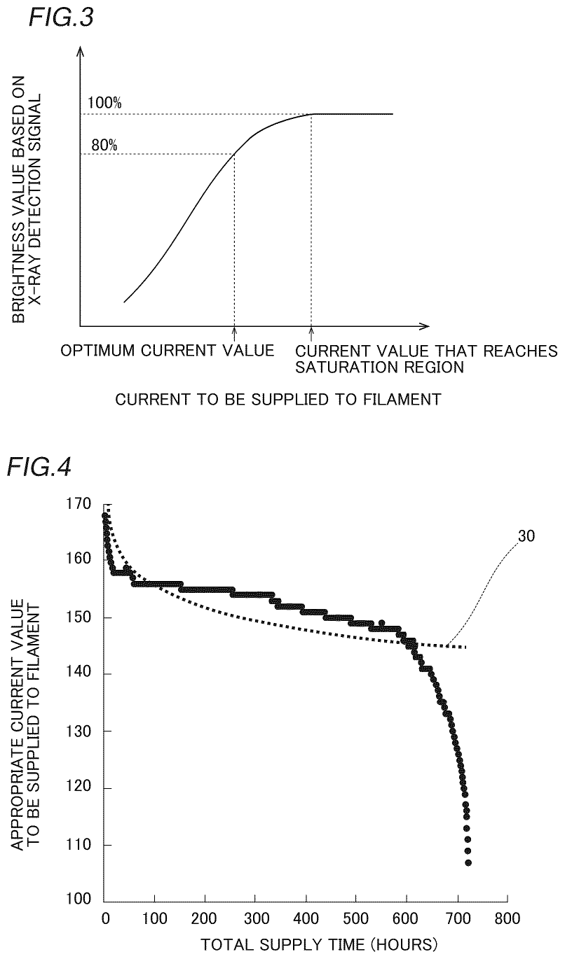

In the process to search for an appropriate current value, the controller 21 sequentially increases the current to be supplied to the filament 19 via the second power supply circuit 18, and acquires a change in a brightness value based on an X-ray detection signal acquired by the X-ray detector 2 in response to the increase in the current to be supplied to the filament 19. FIG. 3 is a diagram showing the relationship between the current to be supplied to the filament 19 and the X-ray brightness. When the current to be supplied to the filament 19 is small, the brightness value based on the X-ray detection signal increases as the current to be supplied to the filament 19 increases. Then, as the current to be supplied to the filament 19 increases, the amount of change in the brightness value based on the X-ray detection signal eventually becomes minute, and the brightness value based on the X-ray detection signal tends to reach a saturation region. When a current exceeding a current value that reaches the saturation region is supplied to the filament 19, the X-ray brightness remains substantially unchanged. However, the filament 19 becomes hotter such that accelerating evaporation and/or consumption of the filament 19 is accelerated, and thus the life of the filament 19 is shortened.

When the brightness value based on the X-ray detection signal is measured while the current to be supplied to the filament 19 is sequentially increased, for example, the controller 21 determines that the current value has reached the saturation region when the measured values of several successive measurement points fall within a preset allowable fluctuation range. Then, the controller 21 acquires, as the appropriate current value, a current value that is 80% of the current value at the time at which the current value reaches the saturation region.

The process to search for an appropriate current value requires acquiring a brightness value based on an X-ray detection signal in a state in which the subject 90 does not appear in the X-ray image. Therefore, in the above process, in a state in which the subject 90 is not placed on the stage, the brightness value based on the X-ray detection signal acquired by the X-ray detector 2 is acquired while the current to be supplied to the filament 19 is sequentially increased. Thus, while the above process is performed, X-ray imaging for non-destructive inspection in which the subject 90 is placed on the stage is interrupted.

### Adjustment of Current to Be Supplied to Filament for Up to First Total Supply Time

The controller 21 adjusts the current to be supplied to the filament 19 based on the appropriate current value acquired by the process to search for an appropriate current value for up to the first total supply time.

The controller 21 acquires an appropriate current value by the process to search for an appropriate current value at each predetermined third total supply time interval for up to the first total supply time from the start of use of the filament 19. The predetermined third total supply time interval is a 24-hour (total supply time) interval, for example. The predetermined third total supply time interval is not limited to 24 hours, but may be shorter than 24 hours or longer than 24 hours.

When the diameter of the diaphragm aperture 15a provided in the aperture 15 is smaller, the X-ray focus of the target 10 becomes smaller as compared with a case in which the diameter of the diaphragm aperture 15a is larger. Therefore, a direction in which the X-rays 9 are radiated is limited such that an X-ray image with improved resolution and clearer contours can be generated, but an X-ray image with a low brightness value is generated due to a reduced amount of X-rays.

Therefore, in order to increase the brightness value, an offset value is set in advance to finely adjust the appropriate current value according to the diameter of the diaphragm aperture 15a provided in the aperture 15. The storage 22 stores the offset value table in which the diameter of the diaphragm aperture 15a provided in the aperture 15 and an offset value for the appropriate current value acquired by the process to search for an appropriate current value are associated with each other. The controller 21 adjusts the current to be supplied to the filament 19 based on the appropriate current value acquired by the above process and the offset value based on the diameter of the diaphragm aperture 15a provided in the aperture 15 by referring to the offset value table. Specifically, the controller 21 adds the offset value to the acquired appropriate current value to adjust the current to be supplied to the filament 19. That is, the controller 21 acquires an appropriate current value by the above process at each predetermined third total supply time interval for up to the first total supply time from the start of use of the filament 19, and adds the offset value to the acquired appropriate current value to adjust the current to be supplied to the filament 19.

### Current Information

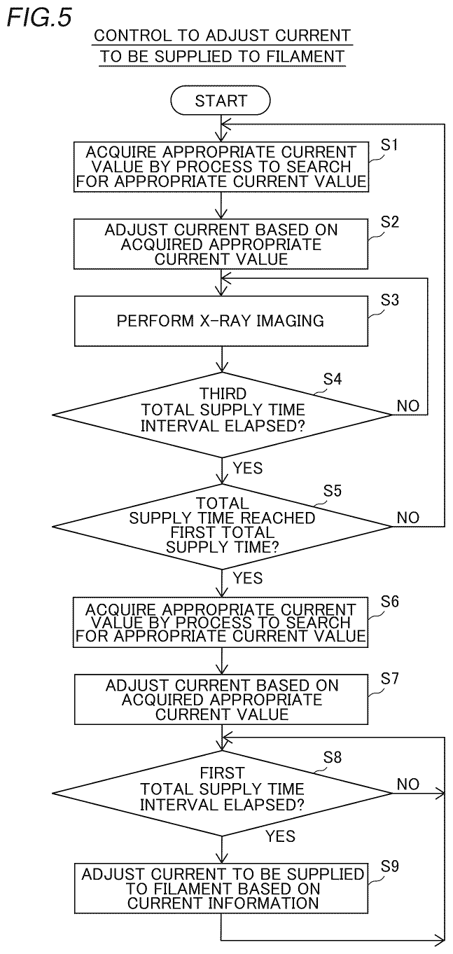

The current information 30 is now described with reference to FIG. 4.

The current information 30 includes information in which a total supply time of a current to be supplied to the filament 19 to emit electrons from the filament 19 is associated with an appropriate current value to be supplied to the filament 19 to emit electrons from the filament 19. Specifically, the current information 30 includes information in which a total supply time is associated with an appropriate current value acquired based on a brightness value based on an X-ray detection signal detected by the X-ray detector 2 while a current is supplied to the filament 19 and the current to be supplied is increased for each predetermined second total supply time interval. The current information 30 is acquired in advance and stored in the storage 22. The current information 30 may be stored in the storage 22 before shipment of the X-ray imaging apparatus 100 or after shipment of the X-ray imaging apparatus 100. The current information 30 stored in the storage 22 may be updatable or replaceable with new current information 30.

The current information 30 includes information about an appropriate current value acquired by the process to search for an appropriate current value for the total supply time of the current to be supplied to the filament 19, which is acquired based on actual measurement data. Specifically, the current information 30 is an approximate curve calculated based on plot points plotted on a graph as appropriate current values for the total supply time in the graph in which the vertical axis represents information about appropriate current values for the total supply time to be supplied to the filament 19 and the horizontal axis represents the total supply time of the current to be supplied to the filament 19.

The process to search for an appropriate current value, which is performed when the current information 30 is acquired in advance, is performed at the predetermined second total supply time interval. That is, an appropriate current value is acquired by the above process, which is performed at each predetermined second total supply time interval. The predetermined second total supply time interval is a 2-hour (total supply time) interval, for example. The predetermined second total supply time interval is not limited to a 2-hour interval, but may be an interval shorter than two hours or longer than two hours. For example, the predetermined second total supply time interval may be a 0.5-hour interval, a 1-hour interval, a 4-hour interval, or an 8-hour interval.

When the current information 30 is acquired, actual measurement data in which the filament 19 was not cut until the predicted maximum usable time of the filament 19 (the predicted life of the filament 19) was reached is used. When the current information 30 is acquired, actual measurement data in which the filament 19 was cut and became unusable before the predicted maximum usable time of the filament 19 was reached is not used.

An initial value is set as an appropriate current value for the total supply time acquired by the first process to search for an appropriate current value, which is performed when the current information 30 is acquired in advance. As an example, the initial value as the acquired appropriate current value for the total supply time is set to “160”. The initial value is not limited to “160”.

Then, an approximate curve is calculated for a plot representing the appropriate current values acquired by the process to search for an appropriate current value performed at each predetermined second total supply time interval. The calculated approximate curve is stored in the storage 22 as the current information 30.

Two pieces of current information 30 are acquired: current information 30 acquired based on actual measurement data acquired under conditions in which the tube voltage of the X-ray tube 1 is 120 kV and the tube current is 50 μA, and current information 30 acquired based on actual measurement data acquired under conditions in which the tube voltage of the X-ray tube 1 is 120 kV and the tube current is 100 μA. The two pieces of acquired current information 30 are stored in the storage 22.

### Adjustment of Current to Be Supplied to Filament for Up to Second Total Supply Time After First Total Supply Time

The controller 21 acquires an appropriate current value by the process to search for an appropriate current value during the first total supply time, and adjusts the current to be supplied to the filament 19 based on the appropriate current value acquired during the first total supply time and the current information 30 acquired in advance and stored in the storage 22, during the second total supply time after the first total supply time.

In non-destructive inspection using the X-ray imaging apparatus 100, when X-ray imaging is performed under the same X-ray imaging conditions as those of either of the two pieces of current information 30, the controller 21 adjusts the current to be supplied to the filament 19 based on the current information 30 under the same X-ray imaging conditions during the second total supply time after the first total supply time. Furthermore, in non-destructive inspection using the X-ray imaging apparatus 100, when X-ray imaging is performed using a tube current different from 50 μA and 100 μA, the controller 21 performs a linear interpolation process on the two pieces of current information 30, and adjusts the current to be supplied to the filament 19 based on the linearly interpolated current information 30 during the second total supply time after the first total supply time.

After the appropriate current value is acquired during the first total supply time, the controller 21 acquires a correction value based on the acquired appropriate current value and the appropriate current value for the first total supply time in the current information 30.

The controller 21 acquires the correction value based on, for example, a difference between the appropriate current value acquired during the first total supply time and the appropriate current value for the first total supply time in the current information 30, the diameter of the diaphragm aperture 15a of the aperture 15 of the X-ray tube 1, and the offset value based on the diameter of the diaphragm aperture 15a of the aperture 15. The storage 22 stores the correction value table in which the appropriate current value difference, the diameter of the diaphragm aperture 15a, and the offset value based on the diameter of the diaphragm aperture 15a, which are described above, are associated with the correction value. The controller 21 acquires the correction value by referring to the correction value table. The items associated with the correction value in the correction value table are not limited to the above. The controller 21 may be configured to acquire the correction value by a relational expression in which the correction value is calculated based on the appropriate current value difference, the diameter of the diaphragm aperture 15a, and the offset value based on the diameter of the diaphragm aperture 15a, which are described above, instead of the correction value table.

Then, the controller 21 adjusts the current to be supplied to the filament 19 during the second total supply time based on the acquired correction value and the appropriate current value for the second total supply time in the current information 30. Specifically, the controller 21 adjusts the current to be supplied to the filament 19 by adding or subtracting the acquired correction value to or from the appropriate current value for the second total supply time in the current information 30.

After the first total supply time, the controller 21 adjusts the current to be supplied to the filament 19 at each predetermined first total supply time interval based on the acquired correction value and the appropriate current value in the current information 30. The predetermined first total supply time interval is a 2-hour (total supply time) interval, for example. The predetermined first total supply time interval is not limited to a 2-hour interval, but may be an interval shorter than two hours or longer than two hours.

After the first total supply time, the controller 21 also shortens the first total supply time interval for adjusting the current to be supplied to the filament 19 as the total supply time becomes longer. As an example, the controller 21 uses a 2-hour (total supply time) interval as the predetermined first total supply time interval until a total supply time of 500 hours in which the slope of the appropriate current value acquired by the process to search for an appropriate current value with respect to the total supply time in the actual measurement data is small and changes the predetermined first total supply time interval to a 0.5-hour (total supply time) interval after a total supply time of 500 hours in which the slope is large to adjust the current to be supplied to the filament 19 at each predetermined first total supply time interval. The timing of changing the predetermined first total supply time interval is not limited to a total supply time of 500 hours, but may be shorter than a total supply time of 500 hours or longer than a total supply time of 500 hours. Moreover, the predetermined first total supply time interval may be changed not only once, but also a plurality of times. Furthermore, the changed predetermined first total supply time interval is not limited to a 0.5-hour interval, but may be an interval shorter than 0.5 hours or longer than 0.5 hours.

Therefore, the controller 21 adjusts, at each predetermined first total supply time interval after the first total supply time, the current to be supplied to the filament 19 by adding or subtracting the acquired correction value to or from the appropriate current value for the second total supply time in the current information 30. That is, the controller 21 adjusts the current to be supplied to the filament 19 based on the current information 30 without performing the process to search for an appropriate current value after the first total supply time, and thus the subject 90 placed on the stage does not need to be moved from the stage in adjusting the current to be supplied to the filament 19. Furthermore, the controller 21 can perform the adjustment process of the current to be supplied to the filament 19 in the background. For these reasons, it is not necessary to interrupt X-ray imaging for non-destructive inspection for the adjustment process of the current to be supplied to the filament 19 after the first total supply time.

### Control to Adjust Current to Be Supplied to Filament

Referring to FIG. 5, a control to adjust the current to be supplied to the filament 19 by the controller 21 (X-ray imaging method) is now described. The current information 30 is stored in advance in the storage 22. When an input operation to terminate X-ray imaging is received, a control to adjust the current to be supplied to the filament 19 by the controller 21 and a control of X-ray imaging are terminated. Furthermore, the order of each step can be reversed or each step can be executed simultaneously as long as there is no contradiction between them.

In step S1, the controller 21 acquires an appropriate current value by the process to search for an appropriate current value without the subject 90 placed on the stage, and then advances to step S2.

In step S2, the controller 21 adjusts the current to be supplied to the filament 19 based on the appropriate current value acquired by the above process, and then advances to step S3.

In step S3, the controller 21 performs X-ray imaging with the subject 90 placed on the stage, and then advances to step S4.

In step S4, the controller 21 determines whether or not the predetermined third total supply time interval has elapsed since the above process. When the predetermined third total supply time interval has elapsed since the above process (Yes in step S4), the controller 21 advances to step S5, and when the predetermined third total supply time interval has not elapsed since the above process (No in step S4), the controller 21 returns to step S3.

In step S5, the controller 21 determines whether or not the total supply time from the start of use of the filament 19 has reached the first total supply time. When it has reached the first total supply time (Yes in step S5), the controller 21 advances to step S6, and when it has not reached the first total supply time (No in step S5), the controller 21 returns to step S1.

In step S6, the controller 21 acquires an appropriate current value by the process to search for an appropriate current value without the subject 90 placed on the stage, and then advances to step S7.

In step S7, the controller 21 adjusts the current to be supplied to the filament 19 based on the appropriate current value acquired by the above process, and then advances to step S8.

In step S8, the controller 21 determines whether or not the predetermined first total supply time interval has elapsed since the previous adjustment of the current to be supplied to the filament 19. When the predetermined first total supply time interval has elapsed since the previous adjustment of the current to be supplied to the filament 19 (Yes in step S8), the controller 21 advances to step S9, and when the predetermined first total supply time interval has not elapsed since the previous adjustment of the current to be supplied to the filament 19 (No in step S8), the controller 21 repeats step S8

In step S9, the controller 21 adjusts the current to be supplied to the filament 19 based on the current information 30, and then returns to step S8.

### Advantages of This Embodiment

According to this embodiment, the following advantages are obtained.

In the X-ray imaging apparatus 100 and the method for adjusting the current to be supplied to the filament 19 according to this embodiment, the current information 30 is acquired in advance, in which the total supply time of the current to be supplied to the filament 19 to emit electrons from the filament 19 is associated with the appropriate current value to be supplied to the filament 19 to emit electrons from the filament 19, and after the first total supply time as the predetermined total supply time, the current to be supplied to the filament 19 is adjusted based on the current information 30. Accordingly, after the first total supply time, the current to be supplied to the filament 19 is adjusted based on the current information 30 acquired in advance, without performing the process to acquire an appropriate current value to be supplied to the filament 19 by supplying a current to the filament 19. Therefore, after the first total supply time, the process to supply a current to the filament 19 to search for an appropriate current value is not performed, and thus a current supply to the filament 19 by the above process can be reduced or prevented. Consequently, it is possible to reduce or prevent shortening of the usable life of the filament 19 due to the above process. Furthermore, after the first total supply time, the current to be supplied to the filament 19 can be appropriately adjusted based on the current information 30 acquired in advance without performing the above process.

In the X-ray imaging apparatus 100 according to the above embodiment, the following further advantages are obtained with the following configurations.

That is, in this embodiment, as described above, the controller 21 is configured or programmed to adjust the current to be supplied to the filament 19 based on the appropriate current value acquired by the process to acquire an appropriate current value to be supplied to the filament 19 by supplying a current to the filament 19 for up to the first total supply time, and to adjust the current to be supplied to the filament 19 based on the current information 30 during the second total supply time after the first total supply time. Accordingly, the appropriate current value is not stable for up to the first total supply time, and thus the current to be supplied to the filament 19 can be adjusted based on the appropriate current value acquired by the above process for the filament 19 currently in use. Furthermore, during the second total supply time after the first total supply time, the appropriate current value is stable, and thus the current to be supplied to the filament 19 can be adjusted based on the current information 30 acquired in advance.

In this embodiment, as described above, the controller 21 is configured or programmed to acquire, during the first total supply time, the appropriate current value by the above process, and to adjust, during the second total supply time, the current to be supplied to the filament 19 based on the appropriate current value acquired during the first total supply time and the current information 30. Accordingly, the current to be supplied to the filament 19 can be accurately adjusted based on the appropriate current value actually measured during the first total supply time and acquired by the above process, and the current information 30 acquired in advance.

In this embodiment, as described above, the controller 21 is configured or programmed to acquire the correction value based on the appropriate current value acquired during the first total supply time and the appropriate current value for the first total supply time in the current information 30, and to adjust the current to be supplied to the filament 19 during the second total supply time based on the acquired correction value and the appropriate current value for the second total supply time in the current information 30. Accordingly, based on the acquired correction value and the appropriate current value for the second total supply time in the current information 30, the current to be supplied to the filament 19 can be accurately adjusted during the second total supply time.

In this embodiment, as described above, the controller 21 is configured or programmed to adjust the current to be supplied to the filament 19 based on the appropriate current value in the current information 30 at each predetermined first total supply time interval after the first total supply time. Accordingly, the current to be supplied to the filament 19 is adjusted based on the appropriate current value in the current information 30 at each predetermined first total supply time interval, and thus it is possible to effectively reduce or prevent shortening of the usable life of the filament 19 due to the above process.

In this embodiment, as described above, the controller 21 is configured or programmed to, after the first total supply time, shorten the first total supply time interval for adjusting the current to be supplied to the filament 19 as the total supply time becomes longer. Accordingly, for the filament 19, the wire diameter of which gradually becomes thinner as the total supply time becomes longer, the first total supply time interval is shortened to adjust the current to be supplied to the filament 19, and thus it is possible to more effectively reduce or prevent shortening of the usable life of the filament 19 due to the above process.

In this embodiment, as described above, the current information 30 includes the information about the appropriate current value for the total supply time, which is acquired in advance based on actual measurement data, and the controller 21 is configured or programmed to, after the first total supply time, adjust the current to be supplied to the filament 19 based on the information about the appropriate current value acquired in advance based on the actual measurement data. Accordingly, the current information 30 acquired in advance based on actual measurement data allows the current to be supplied to the filament 19 to be accurately adjusted.

In this embodiment, as described above, the current information 30 includes the information in which the total supply time is associated with the appropriate current value acquired based on the brightness value based on the X-ray detection signal detected by the X-ray detector 2 while a current is supplied to the filament 19 and the current to be supplied is increased for each predetermined second total supply time interval, and the controller 21 is configured or programmed to adjust the current to be supplied to the filament 19 based on the current information 30 after the first total supply time. Accordingly, after the first total supply time, the current to be supplied to the filament 19 can be more accurately adjusted based on the current information 30 in which the total supply time is associated with the appropriate current value.

In this embodiment, as described above, the first total supply time refers to the predicted total supply time required for a fluctuation in the appropriate current value acquired by the above process to stabilize due to the oxide film of the filament 19 peeling off, and the controller 21 is configured or programmed to adjust the current to be supplied to the filament 19 based on the current information 30 after the first total supply time. Accordingly, after the first total supply time required for the fluctuation in the appropriate current value to stabilize, the current to be supplied to the filament 19 can be appropriately adjusted based on the current information 30.

### MODIFIED EXAMPLES

The embodiment disclosed this time must be considered as illustrative in all points and not restrictive. The scope of the present invention is not shown by the above description of the embodiment but by the scope of claims for patent, and all modifications (modified examples) within the meaning and scope equivalent to the scope of claims for patent are further included.

For example, while the X-ray imaging apparatus is used for non-destructive inspection in the aforementioned embodiment, the present invention is not limited to this. For example, the X-ray imaging apparatus may alternatively be configured for medical use. In such a case, a subject is a living body to be inspected.

While there are two pieces of current information in the aforementioned embodiment, the present invention is not limited to this. For example, the current information may alternatively include only one standard piece of current information, or may alternatively include three or more different pieces of current information.

While two pieces of current information are acquired: the current information acquired based on the actual measurement data acquired under the conditions in which the tube voltage of the X-ray tube is 120 kV and the tube current is 50 μA, and the current information acquired based on the actual measurement data acquired under the conditions in which the tube voltage of the X-ray tube is 120 kV and the tube current is 100 μA in the aforementioned embodiment, the present invention is not limited to this. For example, the values of the tube voltage and the tube current under the conditions of the actual measurement data of the current information are not limited to those described above, but the values of the tube voltage and the tube current under the conditions of the actual measurement data of the current information may alternatively be other values.

While the controller adjusts the current to be supplied to the filament based on the appropriate current value acquired by the process to search for the appropriate current value for up to the first total supply time in the aforementioned embodiment, the present invention is not limited to this. For example, the controller may not adjust the current to be supplied to the filament for up to the first total supply time, or may alternatively adjust the current to be supplied to the filament based on an appropriate current value acquired by another known method for up to the first total supply time.

While the controller adjusts the current to be supplied to the filament based on the appropriate current value acquired by the process to search for an appropriate current value during the first total supply time, and the current information in the aforementioned embodiment, the present invention is not limited to this. For example, the controller may alternatively adjust the current to be supplied to the filament based on an appropriate current value acquired by the process before the first total supply time, and the current information.

While the controller acquires the correction value based on the appropriate current value difference, the diameter of the diaphragm aperture, and the offset value, and adjusts the current to be supplied to the filament based on the acquired correction value and the current information in the aforementioned embodiment, the present invention is not limited to this. For example, the controller may alternatively acquire an appropriate current value difference and adjust the current to be supplied to the filament based on the acquired appropriate current value difference and the current information.

While the controller shortens the first total supply time interval as the total supply time becomes longer in the aforementioned embodiment, the present invention is not limited to this. For example, the first total supply time interval after the first total supply time may alternatively be constant.

While the current information includes the information in which the total supply time is associated with the appropriate current value acquired based on the brightness value based on the X-ray detection signal while a current is supplied to the filament and the current to be supplied is increased for each predetermined second total supply time interval in the aforementioned embodiment, the present invention is not limited to this. For example, the appropriate current value in the current information may not be acquired based on the brightness value based on the X-ray detection signal while a current is supplied to the filament and the current to be supplied is increased for each predetermined second total supply time interval.

While the current information is the approximate curve calculated based on the plot points plotted on the graph as the appropriate current values for the total supply time acquired in advance based on the actual measurement data in the aforementioned embodiment, the present invention is not limited to this. For example, the current information may alternatively be a table acquired in advance based on actual measurement data, in which the total supply time is associated with the appropriate current value. Furthermore, for example, the current information may alternatively be configured by inputting data such as X-ray imaging conditions into a learned model that has been created in advance using various machine learning techniques including deep learning, and obtaining the appropriate current value for the total supply time as an output.

### Aspects

It will be appreciated by those skilled in the art that the exemplary embodiments described above are specific examples of the following aspects.

### (Item 1)

An X-ray imaging apparatus comprising:
an X-ray tube including a target and an electron emitter including a filament to emit electrons to the target;
an X-ray detector to detect an X-ray emitted from the X-ray tube;
a storage to store current information acquired in advance, in which a total supply time of a current to be supplied to the filament to emit the electrons from the filament is associated with an appropriate current value to be supplied to the filament to emit the electrons from the filament; and
a controller configured or programmed to perform a control to adjust the current to be supplied to the filament based on the current information after a first total supply time as a predetermined total supply time.

### (Item 2)

The X-ray imaging apparatus according to item 1, wherein the controller is configured or programmed to adjust, for up to the first total supply time, the current to be supplied to the filament based on the appropriate current value acquired by a process to acquire the appropriate current value to be supplied to the filament by supplying a current to the filament, and to adjust, during a second total supply time after the first total supply time, the current to be supplied to the filament based on the current information.

### (Item 3)

The X-ray imaging apparatus according to item 2, wherein the controller is configured or programmed to acquire, during the first total supply time, the appropriate current value by the process, and to adjust, during the second total supply time, the current to be supplied to the filament based on the appropriate current value acquired during the first total supply time and the current information.

### (Item 4)

The X-ray imaging apparatus according to item 3, wherein the controller is configured or programmed to acquire a correction value based on the appropriate current value acquired during the first total supply time and the appropriate current value for the first total supply time in the current information, and to adjust the current to be supplied to the filament during the second total supply time based on an acquired correction value and the appropriate current value for the second total supply time in the current information.

### (Item 5)

The X-ray imaging apparatus according to any one of items 1 to 4, wherein the controller is configured or programmed to adjust the current to be supplied to the filament based on the appropriate current value in the current information at each predetermined first total supply time interval after the first total supply time.

### (Item 6)

The X-ray imaging apparatus according to item 5, wherein the controller is configured or programmed to, after the first total supply time, shorten the first total supply time interval for adjusting the current to be supplied to the filament as the total supply time becomes longer.

### (Item 7)

The X-ray imaging apparatus according to any one of items 1 to 6, wherein
the current information includes information about the appropriate current value for the total supply time, which is acquired in advance based on actual measurement data; and
the controller is configured or programmed to, after the first total supply time, adjust the current to be supplied to the filament based on the information about the appropriate current value acquired in advance based on the actual measurement data.

### (Item 8)

The X-ray imaging apparatus according to item 7, wherein
the current information includes information in which the total supply time is associated with the appropriate current value acquired based on a brightness value based on an X-ray detection signal detected by the X-ray detector while a current is supplied to the filament and the current to be supplied is increased for each predetermined second total supply time interval; and
the controller is configured or programmed to adjust the current to be supplied to the filament based on the current information after the first total supply time.

### (Item 9)

The X-ray imaging apparatus according to any one of items 2 to 4, wherein
the first total supply time refers to a predicted total supply time required for a fluctuation in the appropriate current value acquired by the process to stabilize due to an oxide film of the filament peeling off; and
the controller is configured or programmed to adjust the current to be supplied to the filament based on the current information after the first total supply time.

### (Item 10)

A method for adjusting a current to be supplied to a filament in an X-ray imaging apparatus including an X-ray tube including a target and an electron emitter including a filament to emit electrons to the target, the method comprising:
acquiring current information in advance, in which a total supply time of the current to be supplied to the filament to emit the electrons from the filament is associated with an appropriate current value to be supplied to the filament to emit the electrons from the filament; and
adjusting the current to be supplied to the filament based on the current information after a first total supply time as a predetermined total supply time.

What is claimed is:

1. An X-ray imaging apparatus comprising:
an X-ray tube including a target and an electron emitter including a filament to emit electrons to the target;
an X-ray detector to detect X-rays emitted from the X-ray tube;
a storage to store current information acquired in advance, in which a total supply time of a current to be supplied to the filament to emit the electrons from the filament is associated with an appropriate current value to be supplied to the filament to emit the electrons from the filament; and
a controller configured or programmed to perform a control to adjust the current to be supplied to the filament based on the current information after a first total supply time as a predetermined total supply time.

2. The X-ray imaging apparatus according to claim 1, wherein the controller is configured or programmed to adjust, for up to the first total supply time, the current to be supplied to the filament based on the appropriate current value acquired by a process to acquire the appropriate current value to be supplied to the filament by supplying a current to the filament, and to adjust, during a second total supply time after the first total supply time, the current to be supplied to the filament based on the current information.

3. The X-ray imaging apparatus according to claim 2, wherein the controller is configured or programmed to acquire, during the first total supply time, the appropriate current value by the process, and to adjust, during the second total supply time, the current to be supplied to the filament based on the appropriate current value acquired during the first total supply time and the current information.

4. The X-ray imaging apparatus according to claim 3, wherein the controller is configured or programmed to acquire a correction value based on the appropriate current value acquired during the first total supply time and the appropriate current value for the first total supply time in the current information, and to adjust the current to be supplied to the filament during the second total supply time based on an acquired correction value and the appropriate current value for the second total supply time in the current information.

5. The X-ray imaging apparatus according to claim 1, wherein the controller is configured or programmed to adjust the current to be supplied to the filament based on the appropriate current value in the current information at each predetermined first total supply time interval after the first total supply time.

6. The X-ray imaging apparatus according to claim 5, wherein the controller is configured or programmed to, after the first total supply time, shorten the first total supply time interval for adjusting the current to be supplied to the filament as the total supply time becomes longer.

7. The X-ray imaging apparatus according to claim 1, wherein
the current information includes information about the appropriate current value for the total supply time, which is acquired in advance based on actual measurement data; and
the controller is configured or programmed to, after the first total supply time, adjust the current to be supplied to the filament based on the information about the appropriate current value acquired in advance based on the actual measurement data.

8. The X-ray imaging apparatus according to claim 7, wherein
the current information includes information in which the total supply time is associated with the appropriate current value acquired based on a brightness value based on an X-ray detection signal detected by the X-ray detector while a current is supplied to the filament and the current to be supplied is increased for each predetermined second total supply time interval; and
the controller is configured or programmed to adjust the current to be supplied to the filament based on the current information after the first total supply time.

9. The X-ray imaging apparatus according to claim 2, wherein
the first total supply time refers to a predicted total supply time required for a fluctuation in the appropriate current value acquired by the process to stabilize due to an oxide film of the filament peeling off; and
the controller is configured or programmed to adjust the current to be supplied to the filament based on the current information after the first total supply time.

10. A method for adjusting a current to be supplied to a filament in an X-ray imaging apparatus including an X-ray tube including a target and an electron emitter including a filament to emit electrons to the target, the method comprising:
acquiring current information in advance, in which a total supply time of the current to be supplied to the filament to emit the electrons from the filament is associated with an appropriate current value to be supplied to the filament to emit the electrons from the filament; and
adjusting the current to be supplied to the filament based on the current information after a first total supply time as a predetermined total supply time.
